(19) United States
(12) Patent Application Publication
(10) Pub. No.: US 2025/0345079 A1
(43) Pub. Date: Nov. 13, 2025

(54) NAVIGATION TOOL FOR CATHETERS

(71) Applicant: NEURAVI LIMITED, GALWAY (IE)

(72) Inventors: ANNALISA SMULLIN, GALWAY (IE); KARL KEATING, GALWAY (IE)

(73) Assignee: NEURAVI LIMITED, GALWAY (IE)

(21) Appl. No.: 18/660,523

(22) Filed: May 10, 2024

(51) Int. Cl. A61B 17/22; A61B 17/00; A61B 90/00

(52) U.S. Cl. CPC A61B 17/22031; A61B 17/00234; A61B 90/39; A61B 2017/00238; A61B 2017/003; A61B 2017/00367; A61B 2017/00477; A61B 2017/00557; A61B 2017/00836; A61B 2017/00853; A61B 2017/00867; A61B 2017/00946; A61B 2017/22034; A61B 2090/3966

(57) ABSTRACT

A navigation tool including a proximal portion, an enlarged portion, a distal portion, and a lumen. The proximal portion, enlarged portion, and distal portion can have a first outer diameter, second outer diameter, and third outer diameter, respectively. The first outer diameter can be smaller than the second outer diameter. The third outer diameter can be smaller than the first outer diameter. The lumen, passing through the proximal portion, enlarged portion, and distal portion, can have an inner diameter smaller than the third outer diameter. The enlarged portion can be configured to not anchor the navigation tool to vasculature within a patient. The distal portion can be configured to navigate the vasculature within the patient aided at least in part by the enlarged portion. At least a portion of the enlarged portion and distal portion can be disposed within vasculature of the patient.

### FIELD

The present technology relates generally to medical instruments, and more particularly, to a navigation tool for catheters to enhance navigability and performance.

### BACKGROUND

Various types of catheters have been developed for intravenous interventions within the body of a patient. Under any circumstance, the primary evaluative measure of any catheter is its ability to course through patient anatomy to access a targeted treatment site. Once the catheter reaches the targeted treatment site, the catheter may also include an implant, a secondary catheter, or ancillary device, placed within its lumen, to execute the designated task. For example, aspiration catheters, which can be used in mechanical thrombectomy procedures, must be able to traverse anatomical challenges such as the ophthalmic ledge. In such cases, current aspiration catheters navigate through patient vasculature to reach an occlusion site, utilizing an ancillary device connected to its proximal end, such as an aspiration pump or syringe to interact with the obfuscation or thrombus.

As the catheter navigates through the patient's vasculature, its performance is appraised primarily based on its efficiency and navigability within said vasculature. Navigability, for example, is a desired characteristic for catheters due to the fact that medical practitioners prefer devices that navigate through patient vasculature atraumatically and easily whilst ensuring effective aspiration. However, navigating patient vasculature can be difficult due to the tortuous nature of the vasculature thereby impacting the performance of the catheter. Therefore, a need yet exists for a navigation tool to provide improved navigability and resultantly enhanced performance for various types of catheters.

### SUMMARY

There is provided, in accordance with an example of the present technology, a navigation tool that may include a proximal portion having a first outer diameter, an enlarged portion having a second outer diameter larger than the first outer diameter and disposed distal of the proximal portion, a distal portion disposed distally of the enlarged portion having a third outer diameter smaller than the first outer diameter, and a lumen passing through the proximal portion, the enlarged portion, and the distal portion having an inner diameter smaller than the third outer diameter. The enlarged portion may be configured to not anchor the navigation tool to vasculature within a patient. The distal portion may be configured to navigate the vasculature within the patient aided at least in part by the enlarged portion. At least a portion of the enlarged portion and the distal portion may be disposed within the vasculature of the patient.

In some examples, the enlarged portion may be further configured to resist deformation of the enlarged portion when at least a portion of the enlarged portion contacts vasculature within the patient.

In some examples, the enlarged portion may further include a first layer and a second layer bonded at one or more locations within the enlarged portion. The first layer and the second layer may include a polymer configured to resist deformation of the enlarged portion when at least a portion of the enlarged portion contacts vasculature within the patient.

In some examples, the distal portion is further configured to traverse at least a portion of a clot within the vasculature of the patient.

In some examples, the enlarged portion may include a first layer, a second layer, and a void disposed therebetween. The first layer, the second layer, and the void disposed therebetween may each be disposed within the enlarged portion. The void between the first layer and the second layer can be configured to allow the enlarged portion to be flexible.

In some examples, the first layer and the second layer are bonded at one or more locations within the enlarged portion.

In some examples, the first layer and the second layer may be oriented in concentric tubular sections with the void disposed therebetween the concentric tubular sections.

In some examples, the void can be filled with at least one of polytetrafluorethylene (PTFE) foam, air, and a vacuum.

In some examples, the void can be flushable with a liquid via one or more flush ports.

In some examples, the enlarged diameter portion may include a shape set Nitinol mesh that can be configured to expand radially when the shape set Nitinol is shortened.

In some examples, the shape set Nitinol mesh can be further configured to expand radially via use of a control wire connected to the shape set Nitinol mesh.

In some examples, the shape set Nitinol mesh can be a trapezoidal shape when fully expanded, wherein a short base of the trapezoidal shape is more proximal than a long base of the trapezoidal shape.

In some examples, the shape set Nitinol mesh can be a spherical shape when fully expanded.

Another embodiment of the present disclosure provides a catheter for insertion into a vasculature of a patient. The catheter may include an outer catheter that can include a catheter lumen with a lumen diameter extending therethrough at least a portion of the outer catheter. The catheter may include a navigation tool that can include an outer enlarged portion with a first diameter larger than the lumen diameter, and an inner smaller portion with a second outer diameter with a second outer diameter less than the lumen diameter. The outer enlarged portion may include one or more sections and can be configured to fully obfuscate the catheter lumen of the outer catheter. The inner smaller portion can be configured to navigate the vasculature within the patient aided at least in part by the outer enlarged portion. At least a portion of the outer enlarged portion and the inner smaller portion can be disposed within the vasculature of the patient.

In some examples, the outer enlarged portion may include a higher stiffness than the navigation tool.

In some examples, the inner smaller portion can be further configured to be oriented proximal a clot within the vasculature of the patient.

In some examples, the one or more sections of the outer enlarged portion can be inflatable thereby allowing the outer enlarged portion to be movable by inflating the one or more sections of the outer enlarged portion.

In some examples, the catheter may also include a hub that can be configured to connect the outer enlarged portion and the inner smaller portion. The hub can include a control that can be configured to control the relative position of at least one of the outer enlarged portion and the inner smaller portion with respect to each other.

In some embodiments, the movement of the control in a first direction can move the relative position of at least one of the outer enlarged portion and the inner smaller portion in a first direction.

In some embodiments, movement of the control in a second direction can move the relative position of at least one of the outer enlarged portion and the inner smaller portion in a second direction opposite the first direction.

Additional features, functionalities, and applications of the disclosed technology are discussed in more detail herein.

### BRIEF DESCRIPTION OF THE DRAWINGS

FIG. 1A is an illustration of an exemplary navigation tool for a catheter, in accordance with an example of the disclosed technology;

FIG. 1B is an illustration of an exemplary navigation tool for a catheter within a patient's vasculature, in accordance with an example of the disclosed technology;

FIGS. 2Ai and 2B are illustrations of a first embodiment of an enlarged portion of an exemplary navigation tool of a catheter, in accordance with an example of the disclosed technology;

FIG. 2Aii is an illustration of a second embodiment of an enlarged portion of an exemplary navigation tool of a catheter, in accordance with an example of the disclosed technology;

FIGS. 2C-2D are illustrations of a third embodiment of an enlarged portion of an exemplary navigation tool for a catheter, in accordance with an example of the disclosed technology;

FIG. 3A is an illustration of a first embodiment of a Nitinol shape set mesh for an enlarged portion of an exemplary navigation tool before and after expansion, in accordance with an example of the disclosed technology;

FIG. 3B is an illustration of a second embodiment of a Nitinol shape set mesh for an enlarged portion of an exemplary navigation tool before and after expansion, in accordance with an example of the disclosed technology;

FIG. 4 is an illustration of an exemplary catheter and navigation tool within vasculature of a patient, in accordance with an example of the disclosed technology;

FIG. 5 is illustrations of an exemplary catheter with a hub and control for an exemplary navigation tool, in accordance with an example of the disclosed technology;

FIG. 6 is an illustration of an exemplary catheter and navigation tool positioned near a clot within a patient's vasculature, in accordance with an example of the disclosed technology.

### DETAILED DESCRIPTION

The following detailed description should be read with reference to the drawings, in which like elements in different drawings are identically numbered. The drawings, which are not necessarily to scale, depict selected examples and are not intended to limit the scope of the invention. The detailed description illustrates by way of example, not by way of limitation, the principles of the invention. This description will clearly enable one skilled in the art to make and use the invention, and describes several embodiments, adaptations, variations, alternatives, and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g. “about 90%” may refer to the range of values from 71% to 110%. In addition, as used herein, the terms “patient,” “host,” “user,” and “subject” refer to any human or animal subject and are not intended to limit the systems or methods to human use, although use of the subject invention in a human patient represents a preferred embodiment. As well, the term “proximal” indicates a location closer to the operator or physician whereas “distal” indicates a location further away to the operator or physician.

As discussed herein, “physician” can include a doctor, surgeon, technician, scientist, operator, or any other individual or delivery instrumentation associated with delivery of a catheter to a designated treatment site within the body of a subject.

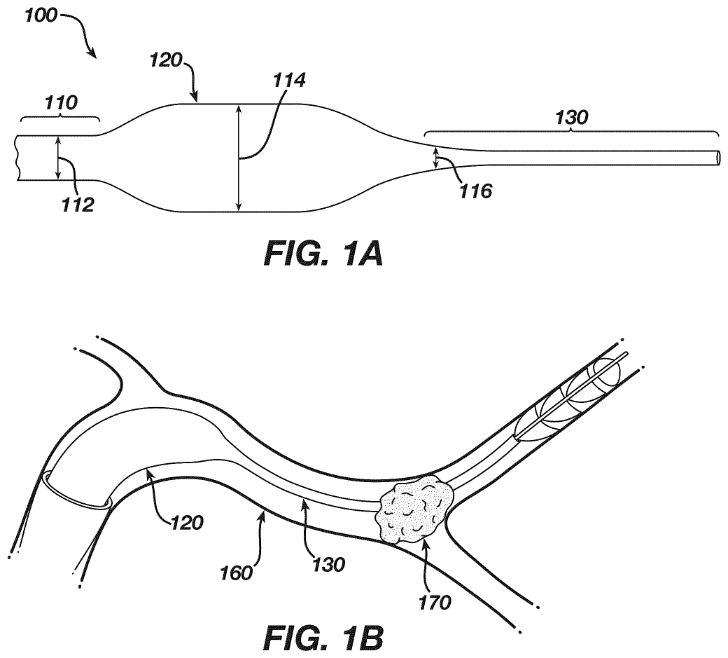

FIG. 1A is an illustration of an exemplary navigation tool (100) for a catheter. The navigation tool (100), as shown in FIG. 1A, can include a proximal portion (110) with a first outer diameter (112). The proximal portion (110) may then transition to an enlarged portion (120) with a second outer diameter (114), which can be larger than the first outer diameter (112). The enlarged portion (120) can be disposed distal of the proximal portion (120) with a non-uniform outer diameter. The enlarged portion (120) may then transition to a distal portion (130) with a third outer diameter (116), which can be smaller than the first outer diameter. The distal portion (130) can be configured to navigate the vasculature (160) within the patient aided at least in part by the enlarged portion (120). The navigation tool (100) can include a lumen (140) passing through the proximal portion (110), the enlarged portion (120), and the distal portion (130). The lumen (140) can include an inner diameter (142) smaller than the third outer diameter (116) of the distal portion (130).

FIG. 1B is an illustration of an exemplary navigation tool (100) for a catheter within a patient's vasculature (160). The navigation tool (100) may be used in conjunction with a catheter, such as a super bore aspiration catheter, to assist a physician in navigating toward a clot (170) within a patient's vasculature (160). To enhance navigability, the enlarged portion (120) of the navigation tool (100) can obfuscate at least a portion of the opening of the super bore aspiration catheter, which can allow the distal portion (130) to traverse the clot (170) within the vasculature (160). In some embodiments, the distal portion (130) can be a microcatheter, which can be implanted with a stent retriever collapsed to be insertable within the inner diameter (142) of the lumen (140) of the navigation tool (100). Once the distal portion (130) traverses the clot (170), as shown in FIG. 1B, the stent retriever can be expanded to engage the clot once the navigation tool (100) and thereby the aspiration catheter are removed from the patient's vasculature (160).

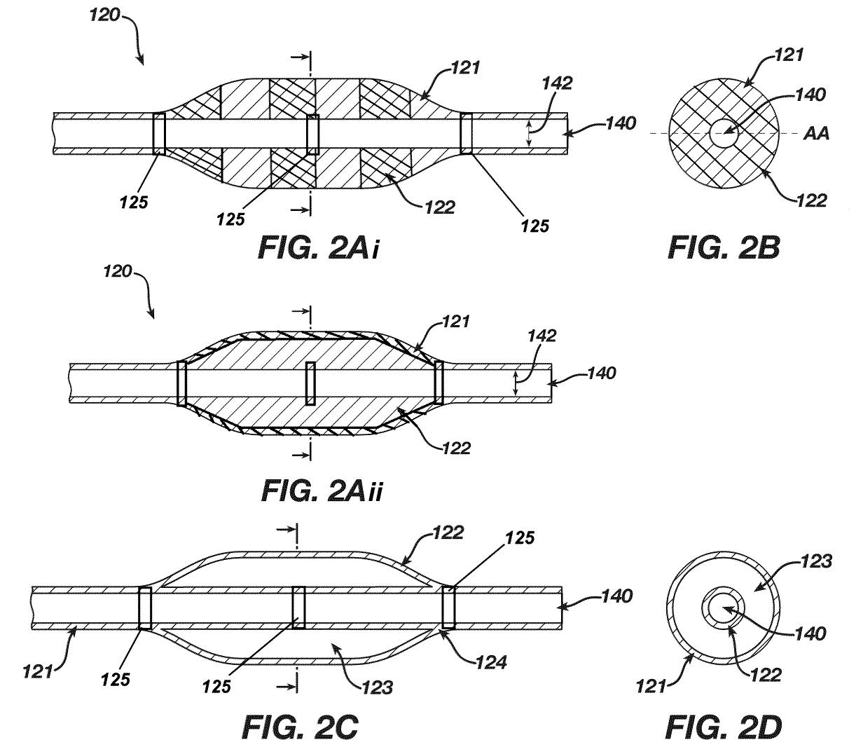

FIGS. 2Ai and 2B are illustrations of a first embodiment of an enlarged portion (120) of an exemplary navigation tool (100) for a catheter. The enlarged portion (120) can be further configured to resist deformation when coming in contact with a patient's vasculature. To resist deformation, the enlarged portion (120) can include a first layer (121) and a second layer (122), that can each include at least a polymer that can be configured to resist deformation of the enlarged portion (120). In some embodiments, as particularly shown in FIG. 2Ai, the first and second layers (121, 122) may be longitudinal/axial layers of polymer with variable durometers. In such embodiments, bands of, for example, softer and harder polymer may be placed next to each other, or in an alternating fashion, such that the softer polymer bands may allow the catheter to flex while the harder polymer bands may help to prevent collapse. For example, in FIG. 2Ai, the first layer (121) may include a softer polymer, e.g., Neusoft 42A, while the second layer (122) may include a harder polymer, e.g., Pebax 55D. Different polymers used may be a mix of softer, mid-range, and harder polymers. For example, a softer or elastomeric polymer may include Chronoprene 40A, Chronoprene 60A, Neusoft 42A, Neusoft 62A, Neusoft 73A, etc.; a mid-range polymer may include Pebax 25D, Pebax 35D, Pebax 45D, etc.; and a harder polymer may include Pebax 55D, Pebax 63D, Pebax 72D, etc. As known in the art, polymers such as polyethylene (PE), have high strength, toughness, and stiffness properties to resist deformation from external loading, such as vasculature (160) of a patient. In some embodiments, both the first layer (121) and the second layer (122) may include the same or dissimilar polymers that can be configured to resist deformation of the enlarged portion (120). In an alternate view, as shown in FIG. 2B, the first layer (121) and the second layer (122) can be understood as a solid radially enlarged axial section with no void or separation between the first layer (121) and second layer (122). In some alternate embodiments, the relative hardness of the first layer (121) and the second layer (122) can be adjusted to obtain a desired flexibility and/or stiffness of the enlarged portion (120).

FIG. 2Aii is an illustration of a second embodiment of an enlarged portion (120) of an exemplary navigation tool (100) for a catheter. FIG. 2Aii may be similar to FIG. 2Ai, except that the first and second layers (121, 122) may be circumferential layers of polymer with variable durometers. For example, as shown in FIG. 2Aii, the first layer (121) may include a harder polymer, while the second layer (122) may include a softer polymer. In some embodiments, the second layer (122) may be thicker than, and disposed internal to, the first layer (121), such that the second layer (122) can help the first layer (121) flex without collapsing or kinking.

FIGS. 2C-2D are illustrations of a third embodiment of an enlarged portion (120) of an exemplary navigation tool (100) for a catheter. In some embodiments, as shown in FIGS. 2C-2D, the enlarged portion can include the first layer (121) and the second layer (122) with a void (123) disposed therebetween, wherein the void (123) can be configured to allow the enlarged portion (120) to be flexible. The void (123), as shown in FIG. 2C, can be filled with at least one of polytetrafluoroethylene (PTFE) foam, air, and a vacuum. In some embodiments, the void (123) can be flushable with a liquid via one or more flush ports. In some scenarios, it can be advantageous for the enlarged portion (120) to be flexible as this can allow the distal portion (130) of the navigation tool (100) to maneuver within the vasculature (160) of a patient toward a targeted clot (170).

For example, in the case of the internal carotid artery (ICA), an aspiration catheter and ancillary devices, such as access aids, microcatheters, and the like, must navigate challenging anatomical structures within the patient's vasculature (160), such as the aortic arch, to enter the ICA. Once within the ICA, the aspiration catheter and ancillary devices must then navigate anatomical structures, such as the ophthalmic ledge, in order to reach the targeted clot (170). With respect to the present disclosure, the navigation tool (100) described herein having a flexible enlarged portion (120) can enhance navigability once deployed from the aspiration catheter while simultaneously reducing the ledge effect. As will be appreciated, mitigation of the ledge effect occurs by reducing the gap between the aspiration catheter and the navigation tool (100) within the aspiration catheter. Given that the enlarged portion (120) can obfuscate at least a portion of the opening of the aspiration catheter, the ledge effect can be effectively mitigated whilst allowing the distal portion (130) to navigate toward the targeted clot (170), thereby increasing performance of the aspiration catheter via utilization of the navigation tool (100) with the flexible enlarged portion (120).

In some embodiments, as shown in FIG. 2D, the first layer (121) and the second layer (122) can be oriented in concentric tubular sections, wherein the void (123) can be disposed therebetween the concentric tubular sections. The lumen (140), as also shown in FIG. 2D, having an inner diameter (142) can also be oriented as an additional concentric tubular section radially within the first layer (121), the second layer (122), and the void (123). The concentric orientation of the first layer (121), the second layer (122), the void (123), and the lumen (140) can be understood as illustrated in FIG. 2C and thus further illustrate how the enlarged portion (120) can be configured to be flexible.

In some embodiments, as shown in FIGS. 2Ai, 2Aii, and 2C, one or more radiopaque marker bands (125) may be used to aid in positioning the catheter. As illustrated, the marker bands (125) may be placed at proximal and distal ends, as well as in the middle, of the enlarged portion (120). Marker bands (125) may be used to allow a user, e.g., a physician, to place the enlarged portion (120) relative to a catheter marker. In some embodiments, a marker band (125) may also be placed on the smaller distal portion (130) of the navigation tool (100), as discussed above with respect to FIG. 1A, such that a physician knows where the distal portion (130) is relative to the outer enlarged portion (120). In some embodiments, marker bands (125) may be made of platinum. In some embodiments, the enlarged portion (121/122) can include soft or hard materials with radiopaque filler (Tungsten, Bismuth, Barium Sulphate). For example, soft elastomeric materials (e.g., Neusoft 42A/62A/73A, Chronoprene 40A, etc.) could allow more flexibility to track and still prevent collapse due to their thickness. Mid-range materials ranging from 25D to 35D may also allow for flexibility, while harder materials (e.g., Pebax 45D to 72D) may be used, but kept shorter to allow tracking across tortuous vessel paths.

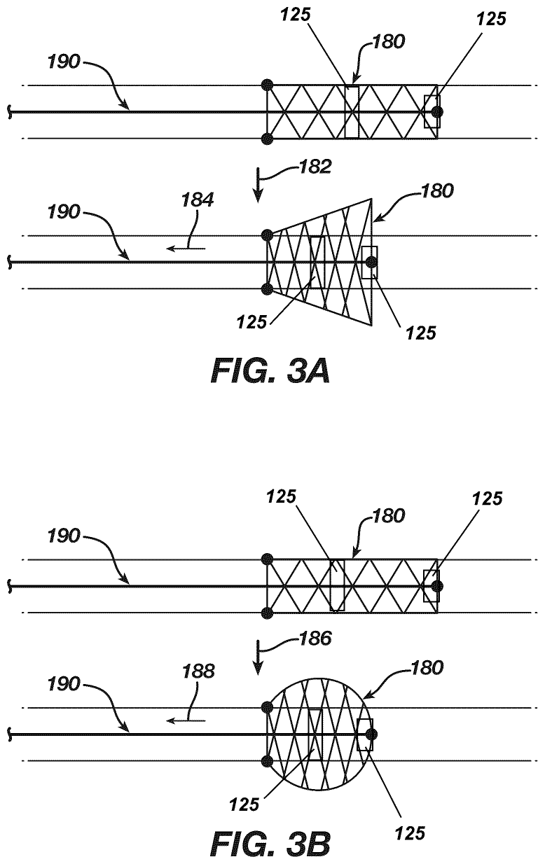

FIG. 3A is an illustration of a first embodiment of a Nitinol shape set mesh (180) for an enlarged portion (120) of an exemplary navigation tool (100) before and after expansion. As known in the art, Nitinol is a metal alloy with “shape-memory” properties, allowing the material to “remember” or change shape when exposed to external stimuli. With respect to the present disclosure, the enlarged portion (120) can include a shape set Nitinol mesh (180) that can be configured to expand radially when the shape set Nitinol (180) is shortened. In some embodiments, the shape set Nitinol mesh (180) can be further configured to expand radially via utilization of a control wire (190) connected to the shape set Nitinol mesh (180). For illustrative purposes, the arrow (182) between the illustrations in FIG. 3A can be understood to show the transition between a pre-expanded state of the shape set Nitinol mesh (180) and a fully expanded state of the shape set Nitinol mesh (180). For example, as shown in the top illustration of FIG. 3A, the shape set Nitinol mesh (180) may be delivered in a pre-expanded form factor when inserted into the patient's vasculature (160). Upon receiving a signal from the control wire (190), as indicated by the arrow (184) in the bottom illustration of FIG. 3A, the shape set Nitinol mesh (180) can become a trapezoidal shape when fully expanded. The trapezoidal shape, when fully expanded, can include a short base that can be more proximal than a long base of the trapezoidal shape, as shown in FIG. 3A. When fully expanded, the shape set Nitinol mesh (180) and thereby the enlarged portion (120) can obfuscate the aspiration catheter to enhance navigability of the navigation tool (100). It should also be appreciated that the second outer diameter (114) of the enlarged portion (120), upon full expansion of the shape set Nitinol mesh (180), can be understood to be larger than the first outer diameter (112), the third outer diameter (116), and the inner diameter (142) of the lumen (140) of the navigation tool (100).

FIG. 3B is an illustration of a second embodiment of the Nitinol shape set mesh (180) for an enlarged portion of an exemplary navigation tool (100) before and after expansion. Similarly to FIG. 3A, for illustrative purposes, the arrow (186) between the illustrations in FIG. 3B can be understood to show the transition between a pre-expanded state of the shape set Nitinol mesh (180) and a fully expanded state of the shape set Nitinol mesh (180). In some embodiments, the shape set Nitinol mesh (180) can be delivered in a pre-expanded form factor when inserted into the patient's vasculature (160) as shown in the top illustration of FIG. 3B. Upon receiving the signal from the control wire (190), indicated by the arrow (188) shown in the bottom illustration of FIG. 3B, the shape set Nitinol mesh (180) can become a spherical shape when fully expanded. It should be appreciated that various resultant shapes for the shape set Nitinol mesh (180) can be realized when fully expanded and the present disclosure is not so limited to the previously mentioned shapes. As mentioned previously, the shape set Nitinol mesh (180), as shown in FIG. 3B, when fully expanded can obfuscate the aspiration catheter to enhance navigability of the navigation tool (100), given that the shape set Nitinol mesh (180) is disposed at the enlarged portion (120) of the navigation tool (100).

In some embodiments, one or more radiopaque marker bands (125), as discussed above with respect to FIGS. 2Ai, 2Aii, and 2C, may be placed on the shaft of the shape set Nitinol mesh (180) and/or on the distal end of the control wire (190), as shown in FIGS. 3A and 3B. In some embodiments, rather than be made of Nitinol, the shape set Nitinol mesh (180) could instead be made from Nitinol drawn filled tubing (DFT) of Nitinol with an inner platinum/tantalum filling.

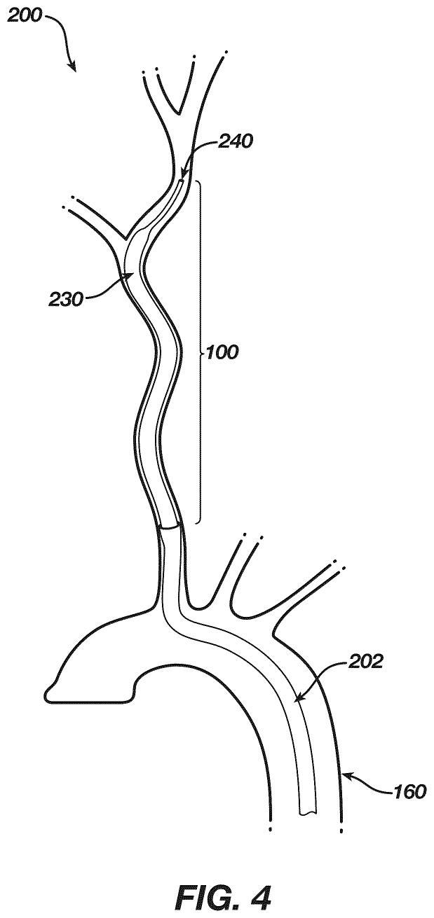

FIG. 4 is an illustration of an exemplary catheter (200) and navigation tool (100) within the vasculature (160) of a patient. For exemplary purposes, the vasculature (160) shown in FIG. 4 can be understood as an exemplary ICA, which can span through the neck and enter the skull of the patient, bifurcating from the common carotid arteries (CCAs) into the internal and external carotids. It should be appreciated, however, that the present disclosure is not so limited to solely application within ICAs. In order to access a targeted ICA, typically a stiff access aid is utilized to support a catheter and thus allow passage through the aortic arch and entrance into the targeted ICA. In an alternate exemplary embodiment of the present disclosure, as shown in FIG. 4, the present technology provides a catheter (200) for insertion into the vasculature (160) of the patient. The catheter (200) can include an outer catheter (202) that can include a catheter lumen (204) having a lumen diameter (206) extending therethrough at least a portion of the outer catheter (202). In some embodiments, the outer catheter (202) comprises a higher stiffness than the navigation tool (100). In other words, the outer enlarged catheter (230) can be configured with a higher stiffness profile and can thereby demonstrate a greater resilience to deformation when contacting the patient's vasculature (160).

The catheter (200) can also include a navigation tool (100) wherein the navigation tool (100) can include an outer enlarged portion (230) with a first outer diameter (232) and an inner smaller portion (240) with a second outer diameter (242) being less than the lumen diameter (206). The outer enlarged portion (230) can include one or more sections (234) that can be configured to fully obfuscate the catheter lumen (204) of the outer catheter (202). The first outer diameter (232) of the outer enlarged portion (230) can be larger than the lumen diameter (206). As shown in FIG. 4, the region where the navigation tool (100) is indicated can be understood to be a transitional point between the outer catheter (202) and the navigational tool (100). The transitional point can also be understood as the point where the navigational tool (100) fully obfuscates the outer catheter lumen (204), as shown in FIG. 4.

The inner smaller portion (240) of the navigation tool (100) can be configured to navigate the vasculature (160) within the patient aided at least in part by the outer enlarged portion (230). In some embodiments, at least a portion of the outer enlarged portion (230) and the inner smaller portion (240) can be disposed within the vasculature (160) of the patient as shown in FIG. 4. By combining the flexible nature of the navigation tool (100) with the stiffness of the outer catheter (202), the inner smaller portion (240), aided by the outer enlarged portion (230) can simplify delivery to the target ICA whilst enhancing navigability and overall performance of the catheter (200).

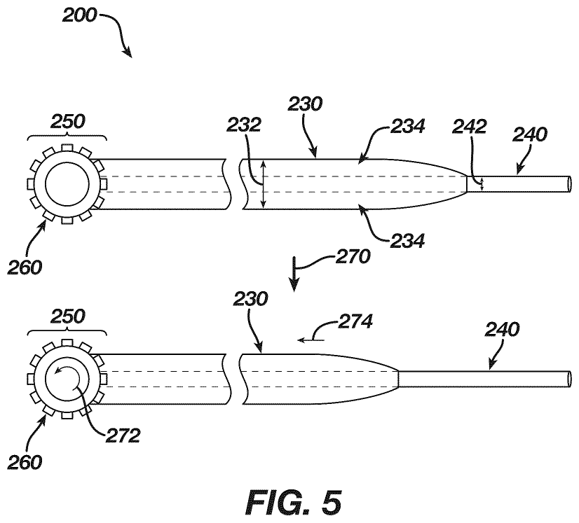

FIG. 5 is illustrations of an exemplary catheter (200) with a hub (250) and control (260) for an exemplary navigation tool (100). In some embodiments, the navigation tool (100) of the catheter (200) can include the hub (250), as pictured in the top illustration of FIG. 5, that can be configured to connect the outer enlarged portion (230) and the inner smaller portion (240). The hub (250) can include the control (260) that can be configured to control the relative position of at least one of the outer enlarged portion (230) and the inner smaller portion (240) with respect to each other. For illustrative purposes, as shown in FIG. 5, the arrow (270) can be understood to illustrate what can occur upon utilization of the control (260) to move the relative position of at least one of the outer enlarged portion (230) and the inner smaller portion (240) with respect to each other. As pictured in the bottom illustration of FIG. 5, movement of the control (260) in a first direction, such as clockwise as indicated by the arrow (272), can move the relative position of at least one of the outer enlarged portion and the inner smaller portion in a first direction, indicated by the arrow (274). Conversely, movement of the control (260) in a second direction, such as counterclockwise for example, can move the relative position of at least one of the outer enlarged portion (230) and the inner smaller portion (240) in a second direction opposite the first direction indicated by the arrow (274). Control over the relative positions of the outer enlarged portion (230) and inner smaller portion (240) can enhance navigability of the catheter (200) as it provides greater control of positioning for the navigation tool (100) within the patient's vasculature.

As mentioned previously, the outer enlarged portion (230) can include one or more sections (234) that can be configured to fully obfuscate the catheter lumen (204) of the outer catheter (202). In some embodiments, the one or more sections (234) of the outer enlarged portion (230) can be inflatable, which can allow the outer enlarged portion (230) to be movable via inflating the one or more sections (234). In other words, the physician can move the relative position of the outer enlarged portion (230) by selecting which of the one or more sections (234) to inflate. Resultantly, control over the relative position of the outer enlarged portion (230) is advantageous as it can aid in navigating the inner smaller portion (240) of the navigation tool (100) and thereby the catheter (200) inside the patient's vasculature (160).

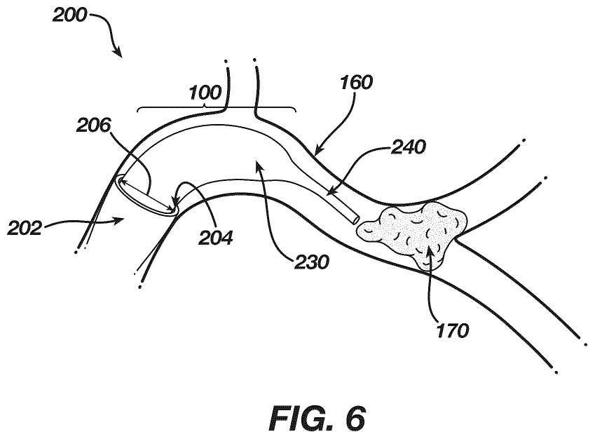

FIG. 6 is an illustration of an exemplary catheter (200) and navigation tool (100) positioned near a clot (170) within a patient's vasculature (160). As shown in FIG. 6, the outer catheter (202) can be disposed within the vasculature (160) of a patient. Upon successful access into the vasculature (160), the navigation tool (100) can fully obfuscate the lumen diameter (204) with the outer enlarged portion (230), which can allow the physician to navigate the inner smaller portion (240) toward the clot (170). In some scenarios, it may be advantageous for the inner smaller portion (240) to not traverse the clot (170) and more preferably be oriented proximal of the clot (170), as pictured in FIG. 6. For example, the positioning of the inner smaller portion (240) at the face of the clot (170) can allow the physician to use direct aspiration techniques to engage the clot (170) for removal from the patient's vasculature (160).

The disclosed technology described herein can be further understood according to the following clauses:

Clause 1: A navigation tool comprising: a proximal portion comprising a first outer diameter; an enlarged portion disposed distal of the proximal portion, the enlarged portion comprising a second outer diameter larger than the first outer diameter and configured to not anchor the navigation tool to vasculature within a patient; a distal portion disposed distally of the enlarged portion and comprising a third outer diameter smaller than the first outer diameter, the distal portion configured to navigate the vasculature within the patient aided at least in part by the enlarged portion; and a lumen, passing through the proximal portion, the enlarged portion, and the distal portion, comprising an inner diameter smaller than the third outer diameter, wherein at least a portion of the enlarged portion and the distal portion are disposed within the vasculature of the patient.

Clause 2: The navigation tool according to Clause 1, wherein the enlarged portion (120) is further configured to resist deformation of the enlarged portion when at least a portion of the enlarged portion contacts vasculature within the patient.

Clause 3: The navigation tool according to Clause 1 or 2, wherein the enlarged portion further comprises a first layer and a second layer, the first layer, and the second layer comprising a polymer configured to resist deformation of the enlarged portion when at least a portion of the enlarged portion contacts vasculature within the patient.

Clause 4: The navigation tool according to any of the preceding Clauses, wherein the distal portion is further configured to traverse at least a portion of a clot within the vasculature of the patient.

Clause 5: The navigation tool according to any of the preceding Clauses, wherein the enlarged portion comprises a first layer, a second layer, and a void disposed therebetween, the first layer, the second layer, and the void disposed therebetween are each disposed within the enlarged portion, wherein the void between the first layer and the second layer is configured to allow the enlarged portion to be flexible.

Clause 6: The navigation tool according to Clause 5, wherein the first layer and the second layer are bonded at one or more locations within the enlarged portion.

Clause 7: The navigation tool according to Clause 5 or 6, wherein the first layer and the second layer are oriented in concentric tubular sections with the void disposed between the concentric tubular sections.

Clause 8: The navigation tool according to any of preceding Clauses 5-7, wherein the void is filled with at least one of polytetrafluoroethylene (PTFE) foam, air, and a vacuum.

Clause 9: The navigation tool according to any of preceding Clauses, wherein the void is flushable with a liquid via one or more flush ports.

Clause 10: The navigation tool according to Clause 1, wherein the enlarged diameter portion comprises a shape set Nitinol mesh, the shape set Nitinol mesh configured to expand radially when the shape set Nitinol mesh is shortened.

Clause 11: The navigation tool according to Clause 10, wherein the shape set Nitinol mesh is a trapezoidal shape when fully expanded, wherein a short base of the trapezoidal shape is more proximal than a long base of the trapezoidal shape.

Clause 12: The navigation tool according to Clause 10 or 11, wherein the shape set Nitinol mesh is further configured to expand radially via use of a control wire connected to the shape set Nitinol mesh.

Clause 13: The navigation tool according to any of preceding Clauses 10-12, wherein the shape set Nitinol mesh is a spherical shape when fully expanded.

Clause 14: A catheter for insertion into vasculature of a patient, the catheter comprising: an outer catheter comprising a catheter lumen with a lumen diameter extending therethrough at least a portion of the outer catheter; and a navigation tool comprising: an outer enlarged portion with a first outer diameter larger than the lumen diameter, the outer enlarged portion comprising one or more sections configured to fully obfuscate the catheter lumen of the outer catheter; and an inner smaller portion with a second outer diameter less than the lumen diameter, the smaller inner portion configured to navigate the vasculature within the patient aided at least in part by the outer enlarged portion, wherein at least a portion of the outer enlarged portion and the inner smaller portion are disposed within the vasculature of the patient.

Clause 15: The catheter according to Clause 14, wherein the outer enlarged catheter comprises a higher stiffness than the navigation tool.

Clause 16: The catheter according to Clause 14, wherein the inner smaller portion is further configured to be oriented proximal a clot within the vasculature of the patient.

Clause 17: The catheter according to Clause 14, wherein the one or more sections of the outer enlarged portion are inflatable thereby allowing the outer enlarged portion to be movable by inflating the one or more sections of the outer enlarged portion.

Clause 18: The catheter according to Clause 14, wherein the catheter further comprises: a hub configured to connect the outer enlarged portion and the inner smaller portion, the hub comprising: a control configured to control the relative position of at least one of the outer enlarged portion and the inner smaller portion with respect to each other.

Clause 19: The catheter according to Clause 18, wherein movement of the control in a first direction moves the relative position of at least one of the outer enlarged portion and the inner smaller portion in a first direction.

Clause 20: The catheter according to Clause 18 or 19, wherein movement of the control in a second direction moves the relative position of at least one of the outer enlarged portion and the inner smaller portion in a second direction opposite the first direction.

Clause 21: The navigation tool according to Clause 1, wherein the enlarged portion further comprises one or more marker bands configured to aid in positioning a catheter in vasculature of a patient.

Clause 22: The navigation tool according to Clause 21, wherein the one or more marker bands comprise one or more radiopaque marker bands.

Clause 23: The navigation tool according to Clause 21 or 22, wherein the one or more marker bands comprise platinum.

Clause 24: The navigation tool according to Clause 10, wherein the enlarged portion further comprises one or more marker bands configured to aid in positioning a catheter in vasculature of a patient, and wherein at least a first marker band of the one or more marker bands is disposed on the shape set Nitinol mesh.

Clause 25: The navigation tool according to Clause 11, wherein the enlarged portion further comprises one or more marker bands configured to aid in positioning a catheter in vasculature of a patient, and wherein at least a first marker band of the one or more marker bands is disposed on the control wire.

Clause 26: The navigation tool according to Clause 1, wherein the enlarged portion further comprises a first layer and a second layer, the first and second layers comprising longitudinal layers of polymer having variable durometers.

Clause 27: The navigation tool according to Clause 26, wherein the first layer comprises a first polymer and the second layer comprises a second polymer that is harder than the first polymer.

Clause 28: The navigation tool according to Clause 1, wherein the enlarged portion further comprises a first layer and a second layer, the first and second layers comprising circumferential layers of polymer having variable durometers.

Any of the examples or embodiments described herein may include various other features in addition to or in lieu of those described above. The teachings, expressions, embodiments, examples, etc. described herein should not be viewed in isolation relative to each other. Various suitable ways in which the teachings herein may be combined should be clear to those skilled in the art in view of the teachings herein.

Having shown and described exemplary embodiments of the subject matter contained herein, further adaptations of the methods and systems described herein may be accomplished by appropriate modifications without departing from the scope of the claims. In addition, where methods and steps described above indicate certain events occurring in certain order, it is intended that certain steps do not have to be performed in the order described but in any order as long as the steps allow the embodiments to function for their intended purposes.

Therefore, to the extent there are variations of the invention, which are within the spirit of the disclosure or equivalent to the inventions found in the claims, it is the intent that this patent will cover those variations as well. Some such modifications should be apparent to those skilled in the art. For instance, the examples, embodiments, geometrics, materials, dimensions, ratios, steps, and the like discussed above are illustrative. Accordingly, the claims should not be limited to the specific details of structure and operation set forth in the written description and drawings.

What is claimed is:

1. A navigation tool comprising:
a proximal portion comprising a first outer diameter;
an enlarged portion disposed distal of the proximal portion, the enlarged portion comprising a second outer diameter larger than the first outer diameter and configured to not anchor the navigation tool to vasculature within a patient;
a distal portion disposed distally of the enlarged portion and comprising a third outer diameter smaller than the first outer diameter, the distal portion configured to navigate the vasculature within the patient aided at least in part by the enlarged portion; and
a lumen, passing through the proximal portion, the enlarged portion, and the distal portion, comprising an inner diameter smaller than the third outer diameter,
wherein at least a portion of the enlarged portion and the distal portion are configured to be disposed within the vasculature of the patient.

2. The navigation tool of claim 1, wherein the enlarged portion is further configured to resist deformation of the enlarged portion when at least a portion of the enlarged portion contacts vasculature within the patient.

3. The navigation tool of claim 2, wherein the enlarged portion further comprises a first layer and a second layer within the enlarged portion, the first layer and the second layer comprising a polymer configured to resist deformation of the enlarged portion when at least a portion of the enlarged portion contacts vasculature within the patient.

4. The navigation tool of claim 1, wherein the distal portion is further configured to traverse at least a portion of a clot within the vasculature of the patient.

5. The navigation tool of claim 1, wherein the enlarged portion comprises a first layer, a second layer, and a void disposed therebetween, the first layer, the second layer, and the void disposed therebetween are each disposed within the enlarged portion, wherein the void between the first layer and the second layer is configured to allow the enlarged portion to be flexible.

6. The navigation tool of claim 5, wherein the first layer and the second layer are bonded at one or more locations within the enlarged portion.

7. The navigation tool of claim 5, wherein the first layer and the second layer are oriented in tubular circular sections with the void disposed therebetween the tubular circular sections.

8. The navigation tool of claim 5, wherein the void is filled with at least one of polytetrafluoroethylene (PTFE) foam, air, and a vacuum.

9. The navigation tool of claim 5, wherein the void is flushable with a liquid via one or more flush ports.

10. The navigation tool of claim 1, wherein the enlarged diameter portion comprises a shape set Nitinol mesh, the shape set Nitinol mesh configured to expand radially when the shape set Nitinol mesh is shortened.

11. The navigation tool of claim 10, wherein the shape set Nitinol mesh is further configured to expand radially via use of a control wire connected to the shape set Nitinol mesh.

12. The navigation tool of claim 10, wherein the shape set Nitinol mesh is a trapezoidal shape when fully expanded, wherein a short base of the trapezoidal shape is more proximal than a long base of the trapezoidal shape.

13. The navigation tool of claim 10, wherein the shape set Nitinol mesh is a spherical shape when fully expanded.

14. A catheter for insertion into vasculature of a patient, the catheter comprising:
an outer catheter comprising a catheter lumen with a lumen diameter extending therethrough at least a portion of the outer catheter; and
a navigation tool comprising:
an outer enlarged portion with a first outer diameter larger than the lumen diameter, the outer enlarged portion comprising one or more sections configured to fully obfuscate the catheter lumen of the outer catheter; and
an inner smaller portion with a second outer diameter less than the lumen diameter, the inner smaller portion configured to navigate the vasculature within the patient aided at least in part by the outer enlarged portion,
wherein at least a portion of the outer enlarged portion and the inner smaller portion are disposed within the vasculature of the patient.

15. The catheter of claim 14, wherein the outer enlarged catheter comprises a higher stiffness than the navigation tool.

16. The catheter of claim 14, wherein the inner smaller portion is further configured to be oriented proximal a clot within the vasculature of the patient.

17. The catheter of claim 14, wherein the one or more sections of the outer enlarged portion are inflatable thereby allowing the outer enlarged portion to be movable by inflating the one or more sections of the outer enlarged portion.

18. The catheter of claim 14, wherein the catheter further comprises
a hub configured to connect the outer enlarged portion and the inner smaller portion, the hub comprising:
a control configured to control the relative position of at least one of the outer enlarged portion and the inner smaller portion with respect to each other.

19. The catheter of claim 18, wherein movement of the control in a first direction moves the relative position of at least one of the outer enlarged portion and the inner smaller portion in a first direction.

20. The catheter of claim 18, wherein movement of the control in a second direction moves the relative position of at least one of the outer enlarged portion and the inner smaller portion in a second direction opposite the first direction.
